(19) United States
(12) Patent Application Publication
(10) Pub. No.: US 2010/0324437 A1
(43) Pub. Date: Dec. 23, 2010

(54) DEVICE AND METHOD FOR ASSESSING PHYSIOLOGICAL PARAMETERS

(76) Inventors: Jenny E. Freeman, Weston, MA (US); Svetlana Panasyuk, Lexington, MA (US)

Correspondence Address: REMENICK PLLC, 1025 THOMAS JEFFERSON STREET, NW, WASHINGTON, DC, 20007, US

(21) Appl. No.: 12/677,216

(22) PCT Filed: Sep. 12, 2008
(86) PCT No.: PCT/US08/76224

§ 371 Date: Mar. 9, 2010

Related U.S. Application Data
(60) Provisional application No. 60/971,642, filed on Sep. 12, 2007.
(60) Provisional application No. 60/973,292, filed on Sep. 18, 2007.

(51) Int. Cl. A61B 5/08

(52) U.S. Cl. 600/529

(57) ABSTRACT

This invention is directed to a device and a method for assessing a subject. The device has at least one impedance measuring element functionally connected to a programmable element, programmed to analyze an impedance measurement, and to provide an assessment of at least one respiratory parameter of the subject. In another embodiment, the device has at least one small-scale motion measuring element. In another embodiment, the device preferably has a high resolution lens, a camera, and a programmable element, which is programmed to analyze at least one physiological parameter of the subject. The physiological parameter is preferably obtained by measuring differential displacements in the camera's field of view. The method according to the present invention involves recording a physiological parameter, analyzing the parameter to make a prediction, and providing an indication of the prediction to a user.

### REFERENCE TO RELATED APPLICATIONS

This application claims priority to U.S. Provisional Application No. 60/971,642 entitled “Stand Off Monitor of Life Signs and Their Variability” filed Sep. 12, 2007, and to U.S. Provisional Application No. 60/973,292 entitled “Stand Off Monitor of Life Signs and Their Variability” filed Sep. 18, 2007, the entirety of each is hereby incorporated by reference.

### BACKGROUND OF THE INVENTION

A. Field of the Invention

This invention is directed to devices and methods for analyzing one or more physiological parameters of a subject based on measurements of small-scale motion, and in particular, repetitive motion that can be quantitatively detected.

B. Description of the Background

Optical assessment of a patient has long been the mainstay of medical evaluation. While many sophisticated techniques such as radar may be used to identify motion at a distance, within the line of sight, much information about an individual's health status is able to be obtained by visual inspection over a short period of time. The skilled clinician subjectively evaluates patient motion and respirations during an examination. A camera system that could provide medically and clinically sufficient spatial and temporal resolution, paired with a software system that could definitively triage a downed warfighter or other patient and provide reliable and rapidly actionable information, would be highly valuable in the field and in the hospital setting as a continuous monitoring device.

Respiration, of course, is critical to survival. In the simplest iteration, presence or absence of breathing is a clear indication of viability or lack thereof. In and of itself, a determination of respiration in a downed soldier, for example, would be useful as a triage tool. As a second tier evaluation, a measurement of respiratory rate would assist in triage and assessment. As a substantial enhancement, however, a device that could also analyze the intensity and variability in the respiratory pattern would have the potential to help identify and quantify the physiological state of a warfighter and/or a patient after injury and help with triage, diagnosis and therapeutic management. While heart rate variability has been extensively studied as a predictor of cardiovascular instability and impending collapse, and while clinicians consider “work of breathing” and evaluation of respiratory pattern as important in their clinical assessment of a critically ill patient, respiratory variability per se in the critically ill or injured patient has received almost no attention. Although there is variation in respiratory pattern associated with sudden infant death syndrome (SIDS) and panic disorder, the available medical critical care data speaks only to evaluation of patients being weaned from mechanical ventilation. A change in respiratory variability, like loss of heart-rate variability, is potentially an indicator of physiologic status.

While certain contact probes record respiratory rate, to date, no device or method has been specifically devised to record or to analyze respiratory variability, to correlate respiratory variability with physiologic condition or viability, or to use respiratory variability to predict impending collapse. Heart rate variability algorithms only report on variations in heart rate, beat to beat. The respiratory rate variability algorithms preferably used according to various embodiments of the present invention incorporate variability in respiratory intensity, rate, and location of respiratory motion. Marked abnormalities in respiration as noted by changes in intensity, in rate, in localization of respiratory effort, or in variability of any of these parameters may provide an early warning of respiratory or cardiovascular failure and may present an opportunity for early intervention. Development of a device to record these changes and creation of algorithms that correlate these respiratory changes with severity of illness or injury would provide not only a useful battlefield tool, but also one of importance in the hospital critical care setting to help evaluate and treat critically ill patients. Use in the clinic or home setting could benefit less critically ill patients that nonetheless would benefit from such monitoring. For example, respiratory rate drops and respirations become “shallow” if a patient is overly narcotized. Respiratory rate and respiratory effort rise with stiff lungs and poor air exchange due to pulmonary edema or other reasons for loss of pulmonary compliance. However, the implications of the rate, which is the only parameter objectively monitored is frequently not identified soon enough to best treat the patient. A system that could provide a real time, quantitative assessment of work of breathing and analyze the trend of respiratory rate, intensity, localization, or variability in any or all of these parameters is needed for early diagnosis and intervention as well as therapeutic monitoring. Such a system is needed to judge the depth of anesthesia, or the adequacy or overdose of narcotic or other pain relieving medications.

### SUMMARY OF THE INVENTION

One embodiment of the present invention relates to a device for assessing a subject, the device comprising: at least one impedance measuring element functionally connected to a programmable element, programmed to analyze an impedance measurement, and to provide an assessment of at least one respiratory parameter of the subject. Preferably, the at least one impedance measuring element is one or more remote probes. Preferably, the one or more remote probes measure body wall movements. Preferably, the one or more remote probes are arranged as a net, vest, or array. Preferably, the one or more probes are placed on the thorax or abdomen of the subject. Preferably, the at least one respiratory parameter is recorded for a duration of 30 seconds; continuously; intermittently; up to at least 10 of the subject's breaths; up to at least 100 of the subject's breaths; or up to at least 1000 of the subject's breaths.

Preferably, the at least one respiratory parameter is selected from the group consisting of the subject's respiratory rate, the subject's respiratory pressure, the subject's respiratory flow, the subject's end tidal CO₂, the subject's sublingual CO₂, intensity of respiration, variability of intensity of respiration, depth of respiration, variability of depth of respiration, localization of respiration, variation in localization of respiration, shape of a respiratory curve, change in shape of a respiratory curve, a respiratory curve based on inhaled volume, a respiratory curve based on exhaled volume, a respiratory curve based on inhaled pressure, a respiratory curve based on exhaled pressure, a respiratory curve based on inhaled flow, a respiratory curve based on exhaled flow, a respiratory curve based on motion of the subject's chest as measured by imaging, a respiratory curve based on motion of the subject's chest as measured by contact sensors placed on the chest, and combinations thereof.

Preferably, the at least one impedance measuring element comprises one or more remote probes, and wherein the programmable element is further programmed to analyze one or more remote probe data sets collected from the one or more remote probes. Preferably, the impedance measurement is based on a plurality of remote probe data sets, and wherein the programmable element is further programmed to enhance at least one of the plurality of remote probe data sets; or to stabilize at least one of the plurality of remote probe data sets; or to analyze each of the plurality of remote probe data sets for dynamic range and signal to noise ratio (SNR) values; or to evaluate and remove global motion; or to build a motion vector map to determine target displacements; or to calculate a differential motion map. Preferably, the at least one respiratory parameter is selected from the group consisting of intensity of an acoustic signal, variability of an acoustic signal. Preferably, the analysis of the at least one respiratory parameter is performed by a method selected from the group consisting of a linear method, a nonlinear method, an entropy method, a similarity of distributions and fractal dimensions method, and combinations thereof.

Preferably, the analysis of the at least one respiratory parameter comprises correlating the at least one respiratory parameter with a predefined respiratory condition. Preferably, the at least one prediction is a prediction is selected from the group consisting of a prediction of the subject's viability, a prediction of injury severity, a prediction of the subject's likelihood of collapsing, a prediction of the subject's likelihood of suffering respiratory failure, a prediction of the subject's depth of anesthesia, a prediction of the subject's drug dosage level, a prediction of the subject's likelihood of cardiopulmonary failure, a prediction of the likelihood of equipment failure for equipment associated with treating the patient, and combinations thereof.

Another embodiment of the present invention is directed to a device for assessing a subject, wherein the device comprises at least one small-scale motion measuring element functionally connected to a programmable element, programmed to analyze a small-scale motion measurement and to provide an assessment of the subject based on the small-scale motion measurement. The at least one small-scale motion measuring element preferably comprises a high resolution lens functionally connected to a camera. The at least one small-scale motion measuring element preferably comprises one or more remote probes.

Preferably, the one or more remote probes measure body wall movements. Particularly preferably, the body wall movements are movements of the subject's thorax, movements of the subject's neck and thorax, movements of the subject's abdomen and thorax, movements of the subject's abdomen, thorax, and neck, movements of the subject's abdomen, thorax, and extremities, and/or movements of the subject's thorax and extremities.

Preferably, the one or more remote probes measure impedance of the subject, and the programmable element is further programmed to analyze an impedance measurement and to provide the assessment of the subject based on the small-scale motion measurement and the impedance measurement. Preferably, the one or more remote probes comprise an accelerometer. Preferably, the one or more remote probes measure an acoustic signal in different regions of the subject's body that reflect pulmonary air exchange Particularly preferably, the one or more remote probes deliver respiratory information, which includes, but is not limited to depth of respiration, intensity of respiration, rate of respiration, and/or localization of respiration. Particularly preferably, the one or more remote probes deliver information as to variability of depth of respiration, variability of intensity of respiration, variability of rate of respiration, and/or variability of localization of respiration.

Preferably the remote probes measure impedance and transport the measurements wirelessly for the analysis of respiratory rate. Particularly preferably, impedance is measured and respiratory rate is analyzed on the subject. It is also preferable to measure the respiratory rate together with optical motion measurements and to provide results after data fusion.

Preferably, the at least one small-scale motion measuring element comprises a plurality of remote probes, and the programmable element is further programmed to analyze one or more remote probe data sets collected from the plurality of remote probes. Particularly preferably, the one or more remote probe data sets comprise a measurement of body wall movements of the subject. Particularly preferably, the one or more remote probe data sets comprise a measurement of impedance of the subject. Particularly preferably, the one or more remote probe data sets comprise a measurement of differential displacements of the plurality of remote probes in relation to one another.

Additionally or alternatively, the programmable element is preferably further programmed to analyze an external probe data set collected from an external probe, and a combination of one or more remote probe data sets and the external probe data set provides a measurement of differential displacements of the plurality of remote probes in relation to the external probe.

Preferably, the measurement of differential displacements provides a respiratory rate of the subject. Preferably, the measurement of differential displacements is based on a plurality of remote probe data sets, and the programmable element is further programmed to segment, to enhance, and/or to stabilize at least one of the plurality of remote probe data sets. It is also preferable that the measurement of differential displacements is based on a plurality of remote probe data sets, and the programmable element is further programmed to analyze each of the plurality of remote probe data sets for dynamic range and signal to noise ratio (SNR) values.

Particularly preferably, the measurement of differential displacements is based on a plurality of remote probe data sets, and the programmable element is further programmed to evaluate and remove global motion, to build a motion vector map to determine target displacements, and/or to calculate a differential motion map.

Another embodiment of the invention is a device for assessing a subject, wherein the device comprises a high resolution lens functionally connected to a camera functionally connected to a programmable element, programmed to analyze at least one physiological parameter obtained by measuring differential displacements in a field of view of the camera. Preferably, the device is mounted on a stationary mount, or on a passively gyro-stabilized platform. Particularly preferably, the device is handheld. If the device is handheld, it is preferably stabilized by a gyrostabilizer. Preferably, the device provides sensitivity to measure the differential displacements from a distance of up to 1 meter, 10 meters, 100 meters, and/or 1000 meters.

The at least one physiological parameter obtained by measuring differential displacements in a field of view of the camera is preferably the heart rate of the subject, and/or the respiratory rate of the subject. Preferably, the measurement of differential displacements is based on a plurality of images, and wherein the programmable element is further programmed to segment at least one of the plurality of images. Preferably, the measurement of differential displacements is based on a plurality of images, and the programmable element is further programmed to enhance, and/or to stabilize at least one of the plurality of images. Particularly preferably, the measurement of differential displacements is based on a plurality of images, and the programmable element is further programmed to analyze each of the plurality of images for dynamic range and signal to noise ratio (SNR) values, and to adjust camera gain and exposure based on the dynamic range and SNR values. Preferably, the measurement of differential displacements is based on a plurality of images, and the programmable element is further programmed to evaluate and remove global image motion, to build a motion vector map to determine target displacements, and/or to calculate a differential motion map.

Particularly preferably, the programmable element is further programmed to analyze one or more remote probe data sets collected from a plurality of remote probes. Preferably, the one or more remote probe data sets comprise a measurement of body wall movements of the subject, a measurement of impedance of the subject, and/or a measurement of differential displacements of the plurality of remote probes in relation to one another.

Preferably, the programmable element is further programmed to analyze an external probe data set collected from an external probe, and a combination of one or more remote probe data sets and the external probe data set provides a measurement of differential displacements of the plurality of remote probes in relation to the external probe.

Preferably, the measurement of differential displacements provides a respiratory rate of the subject. Preferably, the measurement of differential displacements is based on a plurality of remote probe data sets, and the programmable element is further programmed to segment, to enhance, and/or to stabilize at least one of the plurality of remote probe data sets. Preferably, the measurement of differential displacements is based on a plurality of remote probe data sets, and the programmable element is further programmed to analyze each of the plurality of remote probe data sets for dynamic range and signal to noise ratio (SNR) values. Preferably, the measurement of differential displacements is based on a plurality of remote probe data sets, and the programmable element is further programmed to evaluate and remove global motion, to build a motion vector map to determine target displacements, and/or to calculate a differential motion map.

Another embodiment of the invention is directed to a method for assessing a subject, wherein the method comprises recording at least one physiological parameter of the subject with a device, wherein the device comprises at least one small-scale motion measuring element functionally connected to a programmable element, programmed to analyze a small-scale motion measurement and to provide an assessment of the subject based on the small-scale motion measurement; analyzing the at least one physiological parameter to make at least one prediction; and providing an indication of the at least one prediction. Preferably, the at least one physiological parameter is recorded for a duration of 30 seconds, continuously, intermittently, up to at least 10 of the subject's breaths, up to at least 100 of the subject's breaths, or up to at least 1000 of the subject's breaths. Particularly preferably, the method further comprises utilizing previous data to improve data acquisition and data analysis.

Preferably, the at least one physiological parameter is obtained by measuring differential displacements in a field of view of a camera. Measuring differential displacements preferably comprises obtaining a plurality of images of the thorax or abdomen of the subject and recording each of the plurality of images as a respiratory parameter. Particularly preferably, the at least one physiological parameter is obtained by measuring differential displacements of a plurality of probes. Measuring differential displacements preferably comprises obtaining a plurality of data sets from the plurality of probes and recording each of the plurality of data sets as a respiratory parameter.

Preferably, the step of analyzing the at least one physiological parameter of the subject further comprises segmenting, and/or enhancing the at least one physiological parameter. Preferably, the method further comprises analyzing the at least one physiological parameter for dynamic range and signal to noise ratio (SNR) values. Preferably, the step of analyzing the at least one physiological parameter of the subject further comprises evaluating and removing global motion, building a motion vector map to determine target displacements, and/or calculating a differential motion map.

Preferably, the at least one physiological parameter is the subject's heart rate, blood pressure, age, temperature, respiratory rate, respiratory pressure, respiratory flow, end tidal CO₂, and/or sublingual CO₂. Particularly preferably, the at least one physiological parameter is intensity of respiration, and/or variability of intensity of respiration. Particularly preferably, the at least one physiological parameter is depth of respiration, and/or variability of depth of respiration. Particularly preferably, the at least one physiological parameter is localization of respiration, and/or variation in localization of respiration. Particularly preferably, the at least one physiological parameter is a shape of a respiratory curve, and/or a change in shape of a respiratory curve. Preferably, the respiratory curve is based on inhaled volume, exhaled volume, inhaled pressure, exhaled pressure, inhaled flow, exhaled flow, motion of the subject's chest as measured by imaging, and/or motion of the subject's chest as measured by contact sensors placed on the chest. Preferably, the contact sensors are arranged as a net, vest, or array. Preferably, the at least one physiological parameter is intensity of an acoustic signal. Preferably, the acoustic signal is breath sounds. Preferably, the at least one physiological parameter is variability of an acoustic signal.

Preferably, the analysis of the at least one physiological parameter is performed by a linear method, a nonlinear method, an entropy method, and/or a similarity of distributions and fractal dimensions method. Preferably, the analysis of the at least one physiological parameter comprises correlating the at least one physiological parameter with a predefined physiological condition. Preferably, the at least one prediction is a prediction of the subject's viability, a prediction of injury severity, or a prediction of the subject's likelihood of collapsing.

Preferably, the at least one prediction is a prediction of the subject's viability, a prediction of injury severity, or a prediction of the subject's likelihood of suffering respiratory failure. Preferably, the at least one prediction is a prediction of the subject's viability, a prediction of injury severity, or a prediction of the subject's depth of anesthesia. Preferably, the at least one prediction is a prediction of the subject's viability, a prediction of injury severity, or a prediction of the subject's drug dosage level. Preferably, the at least one prediction is a prediction of the subject's viability, a prediction of injury severity, or a prediction of the subject's likelihood of cardiopulmonary failure.

Preferably, the at least one prediction is a prediction of the subject's viability, a prediction of injury severity, or a prediction of the likelihood of equipment failure for equipment associated with treating the patient. Preferably, the equipment associated with treating the patient is a ventilator.

Other embodiments and technical advantages of the invention are set forth below and may be apparent from the drawings and the description of the invention which follow, or may be learned from the practice of the invention.

### DESCRIPTION OF THE FIGURES

FIG. 1 shows a device according to one embodiment of the present invention mounted on a stationary mount.

FIG. 2 shows a handheld version of a device equipped with a gyro-stabilization system, an ultra portable PC, and a targeting scope.

FIG. 3 shows a processing flow block diagram illustrating software for detecting and measuring differential displacements in a camera field of view (FOV) or with probes, performing data or image segmentation, and providing statistical displacement precision enhancement.

FIG. 4 shows a traceability diagram demonstrating performance requirements, system components, component hardware parameters and derived parameters and effects.

FIG. 5 shows a plurality of remote probes positioned on a subject.

FIG. 6(a) shows a plot of Thoracic impedance (ohms) over time (seconds).

FIG. 6(b) shows a plot of Thoracic impedance (ohms) over time (seconds) within a narrower range on the y-axis, i.e. the plot is “zoomed-in” with respect to Thoracic impedance.

### DESCRIPTION OF THE INVENTION

Heart rate variability has been extensively studied as a predictor of cardiovascular instability and impending collapse. In addition, clinicians consider the “work of breathing” and evaluation of respiratory pattern as important in their clinical assessment of a critically ill patient. Nevertheless, neither heart rate variability nor respiratory variability per se has received attention as quantitative evaluations. To date, no device or method has been specifically devised to record or analyze, for example, respiratory variability or to correlate respiratory variability with physiologic condition or to use respiratory variability to predict impending collapse.

It has been surprisingly discovered one can diagnose and monitor therapy for a variety of pulmonary and intrathoracic pathologies, such as, but not limited to, pneumothorax, hemothorax, airway obstruction, large airway disease, small airway disease, and pleural effusion. Moreover, it has surprisingly been discovered one can record and analyze respiratory variability, correlate respiratory variability with physiologic condition and/or viability, and use respiratory variability to predict impending collapse. It has also surprisingly been discovered that the information obtained can be analyzed to identify the implications of, for example, a patient's present or varying respiratory rate, respiratory depth, respiratory intensity, and/or respiratory localization to treat the patient rapidly and effectively. The present invention also surprisingly provides a much needed real time assessment of work of breathing and an analysis of the trend of respiratory rate, intensity, or variability in either or both of these parameters to provide early diagnosis and to allow for appropriate intervention.

The devices and methods according to the present invention overcome the problems and disadvantages associated with current methods and tools used to analyze a physiological parameter of a subject. The present invention preferably evaluates qualitatively and quantitatively both total motion and rhythmic or repetitive motion, which are preferably associated with spontaneous movement of a subject, with breathing, or with heart rate. These results are preferably correlated with other physiologic assessments to optimize calibration, instrument placement and settings, data collection programs, lighting and specifics of data collection in different lighting conditions. Preferred embodiments of the present invention provide tools and methods of evaluating and/or triaging subjects, for example, fallen warfighters, hospital patients, clinic or home care patients or injured athletes. Other preferred embodiments of the invention provide an assessment of a specified individual or multiple individuals at a distance of 100 meters. For example, the device preferably provides clear and concise information to medics and other personnel as to whether a fallen soldier is dead or alive. The device is preferably light-weight, robust, inexpensive, and energy efficient.

In a preferred embodiment the hardware is mounted on a stationary platform and is used to collect data over a short period of time. The time period is preferably under five minutes, more preferably under one minute and most preferably under thirty-seconds. It is also preferable to use software to identify motion of an individual that is correlated with signs of life, to include respiratory efforts and spontaneous movements, and suppresses other kinds of motion in the field of view and from camera motion, such as wind disturbance or ground shake.

In another embodiment, a more specific indication of patient condition based on respiratory effort, rate, and variability is provided. In addition to rate, additional variables can be evaluated independently or incorporated into a unified respiratory failure algorithm. Such additional variables include, but are not limited to intensity, depth, localization, and variability.

In other embodiments, different sources of electromagnetic radiation, radar, sonar, or other means are used to define motion of the individual that are used in assessing respiratory variation. Contact probes that sense motion are preferably used to provide data to respiratory variation algorithms, which define patient status or impending cardiopulmonary failure. To obtain highly specific data, close assessment (even as close as one meter or less) are preferably utilized.

A preferred embodiment of the present invention is a device that provides clinically and medically sufficient spatial and temporal resolution, to triage a subject and to generate reliable and actionable information. The present invention preferably relates to visible light systems, however, IR systems for use in dark environments, are also within the scope of the invention. The invention preferably employs gyroscopic active and passive stabilization hardware and image registration techniques to provide a handheld tool for visualization of respiration and respiratory patterns. The present invention preferably integrates heart rate and variability and respiratory rate and variability data to provide detailed information about a subject's physiologic condition.

Heart rate variability only speaks to temporal variation. On the other hand, respiratory variability includes not only a temporal variation, but also an independent variable of intensity. The intensity of a single breath is the rate at which the breath is inhaled. Intensity of respiration is preferably noted between sequential breaths and in power and time series analyses of multiple breaths. In other words, respiratory variability includes variation in respiratory rate [RV-r] and also variation in intensity of respiration [RV-i]. Variation in respiratory rate [RV-r] is similar to r-r interval for heart rate variability assessment. On the other hand, since variation in intensity of respiration [RV-i] is unique to respiratory variability, respiratory variability has no correlation in the assessment of heart rate variability. Variation in respiratory rate [RV-r] and variation in intensity of respiration [RV-i] are preferably measured independently. It is also preferable to use algorithm to incorporate both variation in respiratory rate [RV-r] and variation in intensity of respiration [RV-i] into the analysis or into a readout.

Furthermore, it is particularly preferable to incorporate depth of respiration into the analysis. Depth of respiration is an average of the total volume of air inhaled with each breath. Variation in the depth of respiration is preferably monitored and incorporated into the analysis. It is also particularly preferable to analyze localization of respiration. Incorporation of other scalar variables, such as, but not limited to, heart rate, r-r interval or blood pressure or temperature with the respiratory variation are preferably included in the algorithms to improve their utility. Marked abnormalities or changes in respiration as noted by changes in intensity, depth, localization, rate, or variability are preferably used to provide an early warning of respiratory or cardiovascular failure and may present an opportunity for early intervention.

According to various particularly preferred embodiments of the present invention, assessment of heart rate and heart rate variability are recorded at a distance of from 1 to 1000 meters, more preferably from 1 to 100 meters, most preferably from 10 to 100 meters, or in a close but stand-off mode with the same camera system, with a different imaging system (including, but not limited to IR, radar, near IR, UV, visible light) or with a contact probe. Integration of heart rate and variability and respiratory rate and variability data preferably provide even more information about physiologic condition.

Preferably, the device and method of analysis gives data in situations of different body habitus, in various military dress (including body armor), in various positions and under various lighting conditions. In one embodiment, ambient lighting is utilized. In another embodiment, specific lighting is delivered with a separate or integrated lighting system. In one embodiment, standard visible imaging techniques with data collection via a lens and Complementary Metal-Oxide-Semiconductor (CMOS) or Charge-Coupled Device (CCD) attached to a computer is used. In some circumstances, night vision goggles, infrared technology, or other devices or methods for enhancing light collection in low light settings are preferably used for night applications. Specific wavelengths of light are preferably used to provide enhancement. Image registration, automatic target recognition and other image preprocessing and enhancing techniques are preferably used in one embodiment. Preferably, laser or other methods for situation, targeting, or aligning the device along with zoom lens and automatic focusing and stabilizing techniques are used.

In another embodiment, the hardware and software is designed to optimize the data output and maximize ease of use. A graphical user interface (GUI) preferably provides the user with a report of the patient status. The GUI preferably takes the form of an enhanced image, a numerical grading system, or a red, yellow green light indicator for dead, ill or well. Particularly preferably, quantitative information is also provided for each of the analyzed parameters.

In another embodiment, the motion data from an optical imaging system is integrated with other sensor modalities (such as heart rate or respiratory rate from a contact sensor, or such as radar, ultrasound, infrared or other imaging techniques) to provide a more comprehensive assessment. In another embodiment, improved spurious motion suppression is effected by using a stabilized portable camera platform, signals from gyro sensors and enhanced software algorithm. In another embodiment, motion suppression technologies are utilized to enable use as a hand-held system without the requirement for a stationary mount.

One embodiment of the present invention detects, differentiates, monitors, and measures small-scale motion, such as respiratory motion. Preferably, the invention provides the sensitivity to assess the heart rate and heart rate variability at a distance, or in a close but stand-off mode from chest wall motion or motion of the skin over a pulsatile artery. Preferred embodiments of the present invention optically detect small-scale motion at a significant distance. Preferred embodiments of the present invention also detect small-scale motion by probes that record chest wall movement or impedance measurements.

It has surprisingly been discovered that an inexpensive, turn-key camera-based system that provides medically and clinically sufficient information for medical personnel to assess a fallen warfighter or patient at up to 100 meters is achieved according to the present invention. Such a device observes or otherwise records motion and respiratory activity of an individual, records and processes the information, and presents results in an intuitive form for accurate assessment of viability and severity of injury. In one iteration, the system acquires data over 30-seconds, provides a digitally enhanced, stabilized, and magnified visual presentation of the individual, and derives the amplitude and temporal characteristics of the respiratory pattern and present respiratory data in near real time.

In another embodiment, a form of probe or electrode is placed on the patient to deliver information about respiratory rate, variability, intensity, or spatial distribution or localization via wires or wireless communication (for example, BLUETOOTH®). Preferably, similar information about heart rate and heart rate variability is also provided by this means. Probes can deliver information as to the frequency of respiration and respiratory variation. They can also provide information about the intensity of respiration. Based on probe placement or other means, information about the quality or characteristics or uniformity or non-uniformity of the breaths is obtained. For example, information can be obtained about whether primarily abdominal or chest muscles are involved, and/or about whether retractions of the intercostals spaces or supraclavicular region are taking place. Information can also be obtained about the shape of the curve that relates to airflow, as derived from any specific location, which could include location, velocity, acceleration, jerk or higher derivatives. Such information is preferably used to determine the status of large airways and small airways.

It is particularly preferable to integrate information from sensors placed in various locations on the body, including, for example, the chest, abdomen, extremities, and/or neck. Such integration provides a more complete picture about the airflow and respiratory effort by different muscle groups.

Some of the figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk or the like). Similarly, the programs may be stand alone programs, may be incorporated as subroutines in an operating system, may be functions in an installed imagining software package, and the like. It should be understood that the various embodiments are not limited to the arrangements and instrumentality shown in the drawings.

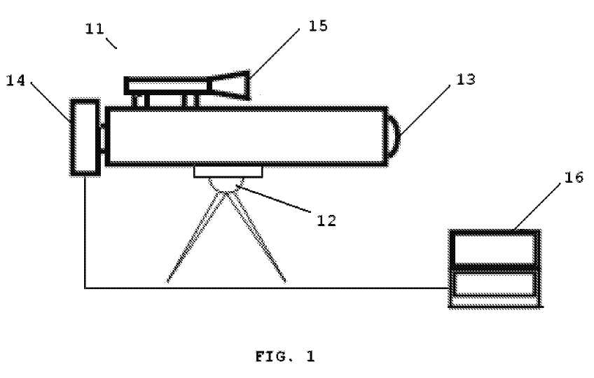

FIG. 1 illustrates a device (11) according to the present invention mounted on a stationary mount (12). Device (11) is equipped with a high resolution lens (13), a telephoto mirror lens. The high resolution lens (13) is functionally connected to a camera (14), a high-resolution USB color camera. Device (11) is also equipped with a targeting scope (15). Finally, camera (14) is functionally connected to programmable element (16), a notebook computer.

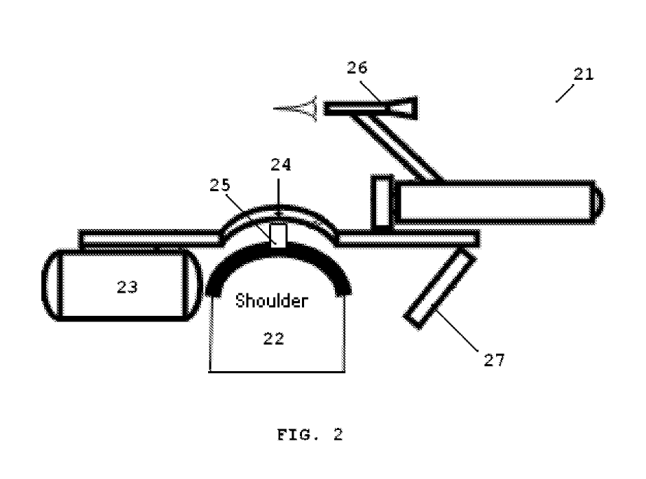

FIG. 2 illustrates a handheld device (21) according to the present invention. Handheld device (21) is shown mounted on a user's shoulder (22) stabilized by gyrostabilizer (23). The center of mass (24) of handheld device (21) is mounted on 2-axis gimbal mount (25). Handheld device (21) is equipped with a targeting scope (26), and a programmable element (27), which, in this case, is an ultra portable personal computer (PC).

Preferably, the handheld device according to the present invention comprises a 500 mm fixed f/8 mirror lens functionally connected to a 2048×1536 color camera with ½″ sensor and 12 fps maximum frame rate at full resolution. Preferably, the camera is functionally connected to a programmable element, which is preferably an ultra portable SONY® VGN-UX1XN PC. The handheld device is preferably stabilized with a KS-6 gyro stabilizer by Kenyon Lab, and preferably equipped with a rifle scope for targeting system. The handheld device according to the present invention preferably has a 4′×3′ full FOV, 2′×1.5′ effective field of view (FOV). The effective field of view is the central part of the full FOV. The effective field of view, as specified above, is preferably maintained during 30-seconds observation period. The handheld device according to the present invention preferably provides motion evaluation precision better than 0.5 mm at 100 m with 10 Hz sample frequency. Preferably, the handheld device according to the present invention provides adequate performance outdoors in reduced light conditions. Preferably, the handheld device according to the present invention weighs less than or equal to 7.25 pounds. The stabilization system preferably weighs less than or equal to 3.25 pounds. The targeting scope preferably weighs less than or equal to 1 pound. The programmable element preferably weighs less than or equal to 1 pound. The camera and lens assembly preferably weighs less than or equal to 2 pounds. Preferably, the handheld device according to the present invention is 18 in×6 in×10 in

The systems illustrated in FIGS. 1 and 2, preferably: discriminate patient motion from global frame motion, caused by factors such as, but not limited to wind pressure, ground shake, and/or concussive blasts; utilize a robust, adaptive camera control algorithm, which controls acquisition parameters to optimize motion resolution precision; employ an image segmentation algorithm, which allows statistical averaging to provide significantly improved displacement measurement precision; and utilize motion analysis software to convert measured displacements of the thorax or abdomen into an assessment of respiratory function. The systems illustrated in FIGS. 1 and 2, preferably permit the average user to maintain 50% of the 4′×3′ field of view throughout a 30 second period. The systems also preferably detect, resolve, monitor and quantify the displacement of surface of the body covers with resolution of ˜1 mm at up to 100 m distance with sample frequency of at least 10 Hz. The systems illustrated in FIGS. 1 and 2, are preferably light, and compact with maximum weight of 10 lb and a size of 2 ft. A preferred device according the present invention provides its own power for 1 hour and is able to be powered/rechargeable from vehicle DC outlet. The systems preferably perform in limited lightning conditions. Additionally the system illustrated in FIG. 2, preferably: employs a hand-held platform that permits the user to easily target and maintain the field of vision (FOV) for the 30 second observation time period; and keeps motion blur during a single frame exposure low enough to avoid image resolution degradation

Using the system described in FIG. 1 or in FIG. 2, the user operates the targeting scope to frame the system field of view (FOV), which preferably includes the lower thorax or upper abdomen of a subject, as well as some static background for differential motion analysis. In the hand held device, shown in FIG. 2, a gyro stabilization system efficiently suppresses ground shake or hand tremor, allowing for smooth targeting and robust focusing. After the system is targeted, a sequence of images from the high resolution camera is preferably processed by software running on the programmable element. Particularly preferably, the device analyzes the sequence of images in real time and notifies the user when a prediction has been made. In other words, as soon as medically and clinically sufficient information is acquired, the instrument notifies the user and stops recording.

Particularly preferably, the software employed in various embodiments of the present invention performs the following sequence of operations to provide personnel with digitally enhanced and numerically processed and qualified information:
1) Images are analyzed for dynamic range and SNR values and camera gain and exposure are adjusted for best performance.
2) Global image motion which corresponds to residual camera jitter is evaluated and removed.
3) A motion vector map is built. Statistically averaging of regions of the image with similar motion vectors allows target displacements to be determined with precision higher than the image resolution. If image noise influences the quality of motion tracking, the camera gain is adjusted to reduce SNR.
4) A differential motion map is calculated which gives relative displacements of the image regions. This step fully suppresses any jitter-related global motion in the image and gives precise measurement of the displacement of the body cover surface.
5) The motion time sequence is analyzed and spatial and temporal motion parameters (including but not limited to amplitude, frequency, regularity) are presented on the visual display in addition to a visually enhanced and stabilized direct target image.

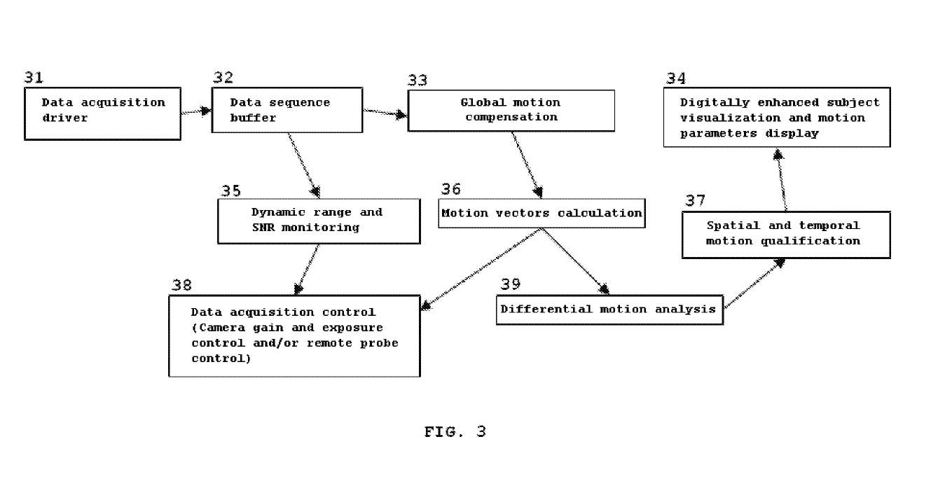

FIG. 3 illustrates software that detects and measures differential displacements in the camera field of view (FOV) or with probes, performs data and/or image segmentation and provides statistical displacement precision enhancement. The software employed in various embodiments of the present invention preferably provides spatial and temporal discrimination to identify motion of an individual correlated with signs of life (signs of life preferably include respiratory efforts and spontaneous movements) and to suppress other kinds of motion in the field of view and from camera motion, such as wind disturbance or ground shake. To provide good low-light system performance the software preferably uses the output of the motion detection algorithm to adjust system parameters automatically to provide real-time optimization of signal-to-noise ratio (SNR) versus dynamic range balance in limited lighting conditions. System parameters adjusted automatically preferably included but are not limited to camera exposure and gain. As shown in FIG. 3, data acquisition driver (31) communicates with data sequence buffer (32). When the data to be analyzed is image data from a camera, data acquisition driver (31) is more aptly called an image acquisition driver and data sequence buffer (32) is more aptly called an image sequence buffer. Data sequence buffer (32) provides global motion compensation (33) and dynamic range and SNR monitoring (35). After Global motion compensation (33), motion vectors calculation (36) can be performed. Both dynamic range and SNR monitoring (35) and motion vectors calculation (36) are used to provide data acquisition control (38). When the data to be analyzed is image data obtained from a camera, data acquisition control (38) preferably comprises camera gain and exposure control. When the data to be analyzed is data from remote probes or from a combination of remote probes and one or more external probes, data acquisition control (38) preferably comprises remote or external probe control. Motion vectors analysis is also used in differential motion analysis (39). Differential motion analysis (39) is used for spatial and temporal motion qualification (37), which in turn is used to generate digitally enhanced subject or target visualization and motion parameters display (34).

Preferably, the device according to the present invention differentiates between alive (breathing) and dead (not breathing). Particularly preferably, it also provides a more specific indication of patient condition based on respiratory rate, effort, and variability. A variety of methods, including standard methods of analyzing time series including linear and nonlinear methods and entropy, similarity of distributions and fractal dimensions useful for analyzing heart rate (R-R interval) variability are preferably applied including entropy, similarity of distributions and fractal dimension. Adaptations of these and new linear and non-linear methods are also preferably employed to analyze the variability in the intensity or depth of breathing.

Preferably the present invention relates a change in impedance to a change in the subject's tissue, a change in the volume of air in the subjects lungs, and/or to a change in the electrical signals from the heart. Particularly preferably, the present invention isolates a respiratory aspect of the impedance measurement. It is also particularly preferable to relate impedance changes to changes in the spatial positioning of probes used to measure impedance. Preferably, the determination of spatial position of the probes is used to generate an image of the subject's body. The resolution of the image depends on the number of probes employed, as each probe preferably represents a single point on a map of the subject's body. Preferably, the invention derives respiratory variability data from changes in impedance, for example, by analyzing changes in the relative positions of the probes over time.

Heart rate variability analyses are preferably performed in time, frequency, time-frequency domains, linear and non-linear methods. These methods include, but are not limited to nonparametric methods including FFT-based PSD (power spectral density), such as Periodogram and Welch; parametric methods including model-based PSD, such as autoregressive (AR) spectrum and autoregressive moving average (ARMA) spectrum; Short Time Fourier Transform (STFT), the Gabor expansion, or the Continuous Wavelet transform; the Poincaré Plot methods and detrended fluctuation analysis (DFA) which quantifies the fractal scaling properties of interval signals; methods of nonextensive and Q-statistics.

The following statistical, geometrical, and frequency measures are preferably analyzed: mean, standard deviations, rms (root mean squares—square root of the mean of the sum of squares of differences between adjacent intervals), covariance structure, entropy, percent, triangular index, morphology, peak frequencies, power spectral density, fractal dimension, correlations, degrees of dispersion, similarity of distribution.

In another embodiment of the present invention, the analysis methods include but are not limited to applying methods of inferential statistics, in particular applying Bayesian inference. Preferably, subjects are grouped according to their attributes, such as, but not limited to heart cycle, respiratory cycle, weight, age, and any other physiological, physical, medical parameters as well as their medical, psychological, etc. history and current state. After evaluating a subject under observation, their probability of belonging to any particular group (cohort) is preferably evaluated and they are assigned accordingly to these groups. Preferably an assessment of the subject's condition is made and provided to a medical professional in the form of a probability, for example, but not limited to a 50% probability that the patient is in “stable state”, a 10% probability that the patient is in “irreversible shock” state, and/or a 20% probability that the patient is in “reversible shock.” Preferably, the patient is kept under continuous monitoring, and all vital signs, including respiratory rate are processed in real-time with an algorithm. As new data become available, the probabilities are preferably re-evaluated and displayed for the medical professional to help him/her assess the efficiency of the medical intervention or to help him/her evaluate the change in patient's state.

Unlike heart rate data, additional information can also be obtained from analysis of the depth of respiration and the variability in depth, which in some iterations allow for a reduction in the number of sequential breaths required for meaningful analysis. The shape and variability in the shape of the respiratory curve is also preferably analyzed. Respiratory curves are preferably based on inhaled volume, exhaled volume (either on a ventilator or other means of collecting exhaled or measuring inhaled gas), inhaled pressure, exhaled pressure, inhaled flow or exhaled flow or on motion of the chest as measured by imaging or by contact sensors placed on the chest.

Probes are preferably placed on the body surface or inside the body on tubes such as, but not limited to, an endotracheal tube, a nasogastric tube, a foley catheter or a line placed in the central blood stream such as a central line or a swanganz catheter. A probe placed within the respiratory circuit either in the patient or outside the patient as part of a mask or nasal canula or ventilator system is preferably used to measure flow or pressure or volume of gas exchange and the variability of these measurements. Preferably, data acquired from sensors affixed to the body surface is analyzed to determine respiratory rate, intensity, depth, and spatial variability. Particularly preferably, impedance electrodes are affixed to specified regions of the body. Preferably such electrodes are similar to electrodes used for continuous patient monitoring in a critical care setting, or are similar to electrodes used in devices that utilize impedance to provide information as to cardiac output.

The respiratory rate variability is preferably analyzed. The variability in respiratory intensity is also preferably analyzed. The variability in respiratory depth and the variability in respiratory localization are also preferably analyzed. Preferably the spatial characteristics of chest wall and abdominal wall motion associated with respiration are also analyzed based on input from probes placed on different parts of the body, including, for example the chest, abdomen, neck, and/or extremities. Additional probes may be placed elsewhere on the body such as the head if impedance, or on the bed or other immobile structure if an accelerometer or other kind of motion detector.

Particularly preferably, combinations of respiratory rate and variability are analyzed. Furthermore, it is preferable to analyze respiratory contents, for example, the concentrations of oxygen, carbon dioxide, nitrogen in the subject's exhaled breath. Combinations of respiratory rate and/or rate variability and spatial location and/or location variability are preferably analyzed. Combinations of respiratory intensity and/or intensity variability and respiratory rate and or rate variability are preferably analyzed. Indeed, it is preferable to analyze any or all combinations or permutations of rate, rate variability, intensity, intensity variability, depth, depth variability, spatial determination of body motion associated with breathing, variability in body motion associated with breathing.

Another embodiment of the present invention involves assessing one dimensional, two dimensional or three dimensional motion of the chest wall or developing a two or three dimensional sensor array or series of images (including, but not limited to optical, x-ray, CT scan, or thermal) to provide in combination an assessment of the three dimensional motion of the body wall or the intrathoracic volume. Such a sensor array is preferably two or more electrodes placed on the body surface. The sensor array transmits data to a device either through a wire or wirelessly via a BLUETOOTH® or other wireless mechanism. All or any aspects of the changes in these three dimensional volumes over time is analyzed independently or in combination. For example, rate, overall volume, change in volume in different intrathoracic regions, changes in shape of the intrathoracic space, changes in position of the surface, changes in position of one part of the surface relative to another are analyzed.

It is preferred that a set of sensors is attached to a critically ill patient, and the position of the sensors is either placed according to a formula, or is recorded via a GPS-like system or via a system like that present in a computer IR mouse. It is also preferred that a vest of sensors is worn by a critically ill patient and the position of the sensors recorded as is done with the position of a computer IR mouse. Accelerometers are preferably used to track such motion.

First, second, third or higher derivatives of chest wall motion or intrathoracic volume are preferably used to assess changes. Motion vectors are analyzed in this context. One sensor shows motion in one direction. Two sensors shows motion in orthogonal directions. Multiple sensors show motion of the irregular surface of the body.

A probe within an endotracheal tube or within a ventilator is preferably used to collect respiratory effort data. Plots of expired oxygen or expired CO₂, pressure of expiration, volume of expiration, pressure of inspiration, or volume of inspiration are preferably used to assess respiratory rate and rate variability and intensity and intensity variability independently or in combination. Data from the expired oxygen or expired CO₂ or pressure or volume of expiration or pressure or volume of inspiration is preferably combined with rate and/or intensity variability to provide additional relevant physiologic data.

One embodiment of the device according to the present invention is a continuous monitor to assess patient status in real time. Preferably, the results are presented in real time on a monitoring screen. Such a device is useful in an intensive or critical care environment. Preferably, the continuous monitoring device comprises cut off values that indicate a change in patient status warranting attention as an early warning of respiratory or cardiovascular collapse.

A closed loop feedback system is preferably established based on data collected from the device. Such data is preferably used to adjust ventilator settings or to adjust medications such as paralytics. Preferably, a patient who would be best served by being totally paralyzed to optimize mechanical ventilation could be monitored and if small spontaneous breaths were occurring, they could be recorded by the device and the physician notified or additional paralytic medication automatically delivered. Alternatively, with another set of patient care imperatives, respiratory motion change could indicate to the physician or automatically trigger an increase in the mechanical ventilation.

While pulse oximetry provides an indication of adequacy of oxygenation, most often respiratory failure is related instead to inadequate respiration and CO₂ retention. According to the present invention data relating to adequacy of respiration and CO₂ retention is preferably combined with other physiologic data such as cardiac output, stroke volume or other impedance derived data to provide additional information as to patient status. The data is particularly preferably combined with heart rate data or heart rate variability data, however, in certain scenarios, the data is preferably combined with other vital sign data such as, but not limited to temperature, or blood pressure.

It is also preferable to combine the data relating to adequacy of respiration and CO₂ retention with acoustic data recorded from a probe placed on the chest wall, a probe placed in the endotrachial tube, a probe placed in a ventilator circuit, a probe placed in the path of respiration, or a probe placed in a mask or nasal cannula. Such acoustic data provides information as to respiratory rate, intensity, and/or acceleration. In particularly preferred embodiments of the present invention, several acoustic sensors are placed to provide greater accuracy of information, to demonstrate differences in air exchange in different parts of the lung, or to demonstrate differences in respiratory motion. Such acoustic differences are preferably analyzed independently. However, in some scenarios, such acoustic readings are integrated with positional sensors, image-based evaluation of chest wall motion or impedance measurements.

In other circumstances, it is preferable to obtain data regarding air flow through an endotrachieal tube in an intubated patient, or to use other means to measure respiratory flow in an awake patient. Such data is analyzed alone or in conjunction with acoustic data, position sensor data, and/or image data. In other circumstances, it is preferable to obtain velocity, acceleration, and/or variability of rate or intensity data from a flow sensor. Such data is analyzed as described for other methods of acquiring data as relating to respiratory rate and intensity.

Other embodiments of the present invention identify respiratory problems and/or identify the progression of respiratory problems to critical levels. Preferably, the parameter that is monitored is respiratory rate. The present invention preferably provides a real time assessment of work of breathing and analyze the trend of respiratory rate, depth intensity or variability in either or all of these parameters would be useful in early diagnosis and intervention.

Whereas thoracic wall excursion is generally evaluated, motion of the abdomen and/or neck is preferably utilized in combination with thoracic motion or independently to provide necessary or supplemental data. Breathing changes to “abdominal breathing” are also monitored under certain circumstances. “Rib retractions” or use of intercostal muscles or supraclavicular muscles to assist respiration are preferably evaluated specifically for presence, intensity and variability. Changes in breath sounds, including what are known clinically as such things as wheezing, or rales are preferably noted by the acoustic sensor. Changes in breath sounds and variability in breath sounds are preferably recorded and the data processed. Such data is preferably used alone or in combination with body surface motion data.

Preferably, the present invention achieves improvements in spurious motion suppression by implementing an actively stabilized portable camera platform, signals from gyro sensors, and enhanced software algorithms Implementing an auto-focusing system further simplifies system operation. The software preferably performs more elaborate diagnostics based on spatial and temporal characteristics of measured displacements and provides a user friendly graphical user interface (GUI) with clear red/yellow/green indicators for rapid assessment of an individual.

Preferably, a user places probes in specified locations on the subject's body, to include the chest and abdomen, or places the probes and allows the system to record their position. Another reference probe or probes are preferably placed on at least one other part of the body, and/or on a non-moving location such as a portion of the patient's bed. As soon as medically and clinically sufficient information is acquired, the instrument notifies the user and stops recording. Alternatively, the device records continuously and the data are analyzed in real-time. The deviations from the original patient's state are preferably noted and displayed to show change of patient's status. The software employed in various embodiments of the present invention preferably performs the following sequence of operations to provide personnel with digitally enhanced and numerically processed and qualified information:
1) Data sets are analyzed for dynamic range & SNR values and gain adjusted for best performance.
2) Corresponding global motion is evaluated and removed.
3) A motion vector map is built. Statistically averaging of regions of the 2D or 3D data set with similar motion vectors allows us to determine target displacements with precision higher than the “image” resolution. If noise influences the quality of motion tracking, the instrument gain is adjusted to reduce SNR.
4) A differential motion map is calculated which gives relative displacements of the body surface regions. This step fully suppresses any global motion and gives precise measurement of the displacement of the body surface.
5) The motion time sequence is analyzed and spatial and temporal motion parameters (amplitude, frequency, regularity etc) are presented on the visual display

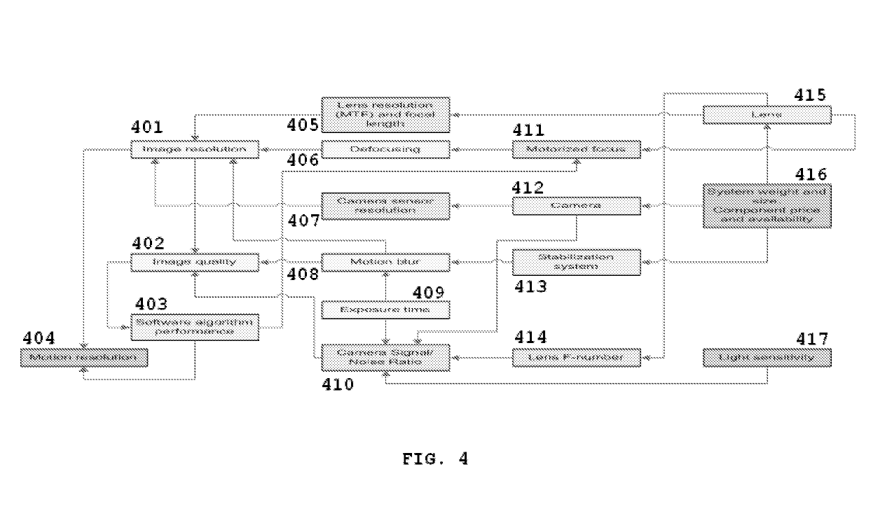

FIG. 4 shows a traceability diagram demonstrating the interrelationships between performance requirements, system components, component hardware parameters and derived parameters and effects. Performance requirements preferably include motion resolution (404), system weight and size, component price and availability (416), and light sensitivity (417). System components preferably include software algorithm performance (403), camera (412), stabilization system (413), and/or lens (415). Component hardware parameters preferably include lens resolution (MTF) and/or focal length (405), camera sensor resolution (407), camera signal/noise ratio (410), motorized focus (411), and/or lens F-number (414). Derived parameters and effects preferably include image resolution (401), image quality (402), defocusing (406), motion blur (408), and/or exposure time (409). The precision of the system in motion evaluation depends on image resolution and motion tracking software performance. The most important parameters affecting resolution are: focal length, camera sensor pixel size, lens resolution and, stabilization of FOV and motion blur due to camera motion and defocusing. To be able to achieve the highest image resolution, the focal length is preferably selected to be as long as possible within imposed weight, size and price limitations. A 500 mm telephoto mirror lens is particularly preferable. While using such a lens, a 1 mm target displacement at 100 m translates to 5 microns displacement in camera sensor space. So as not to be the limiting factor, the camera's pixel size is preferably be smaller than this. Preferable sensors have a pixel size as low as 1.7 microns. A trade-off being that decreased pixel size lowers camera sensitivity. A 3.2 micron pixel size camera sensor is preferable.

Lens resolution is a critical factor because even the best compact 500 mm mirror lenses achieve only about 80 lp/mm MTF resolution, which translates to 12.5 microns resolution element size in sensor space. Thus, it is preferable to employ statistical averaging of the image regions with similar motion vectors (this approach is somewhat analogous to the “centroiding” process used in high-res image acquisition). Because such regions on an image will include thousands of pixels or more, various embodiments of the present invention achieves very significant motion resolution improvement (by a factor of 10 or more) over static image resolution.

Motion blur and FOV stabilization is another critical factor. For a light handheld system without any stabilization, for exposure time on an order of 0.1 sec, the angular standard deviation is preferably on the order of 0.1 degrees, which translates to 900 microns displacement in sensor space. This is certainly unacceptable. Thus, it is preferable to use a KS-6 by Kenyon Labs. This stabilizer has a 3.4″ diameter, a 5.8″ length and weighs 3.25 lbs.

The invention preferably employs a 500 mm focal length lens connected to 2× teleconverter, so it is equivalent to a 1000 mm lens. A mirror lens is preferred which allows for a compact design. The lens is preferably fixed aperture with a preferred F-number 8—with the converter this is equivalent to an F-number of 16, which is preferably used with adequate lighting. The image is preferably projected onto a 6 mm×4 mm Complementary Metal-Oxide-Semiconductor (CMOS) camera with 2048×1536 resolution (3.2×3.2 micron pixels). At a 100 meter distance one pixel corresponds to 3.2 micron×distance/focal length=0.3 mm size on the target, so breathing is easily detected. To estimate motion over large region on the image preferably uses numerical filtering to improve motion resolution and brings the system to a theoretical resolution or better.

To facilitate targeting, the invention preferably employs a targeting scope, for example, a sniper rifle scope. To further facilitate targeting, preferred embodiments of the invention also employ auto-focusing, auto-exposure adjustment, and/or image enhancement algorithms.

Preferred embodiments of the present invention employ existing motion detection and discrimination algorithms. Particularly preferred embodiments employ enhanced motion detection and discrimination algorithms. The software employed according to various embodiments of the present invention preferably provides a convenient graphical user interface (GUI) for visual triage as well as numerical values for estimated motion ranges, and/or frequencies. In addition to direct visual assessment of focusing on PC screen image operator the software preferably provides numerical real-time assessment of focusing quality to facilitate manual focusing. It is particularly preferable to employ a motorized lens and auto focusing algorithm.

Another preferable way to reduce motion blur is to use shorter exposure times for individual frames. A trade-off is that with fixed lens f-number and constant lighting conditions, in order to decrease exposure time, the camera sensor gain has to be increased. After a certain point, the gain increase leads to a significantly increased signal-to-noise ratio (SNR) on the images which influences motion tracking quality. To get the best possible performance, adaptive software control is preferably used to adjust in real-time gain and camera exposure based on motion tracking performance analysis and changing lighting conditions.

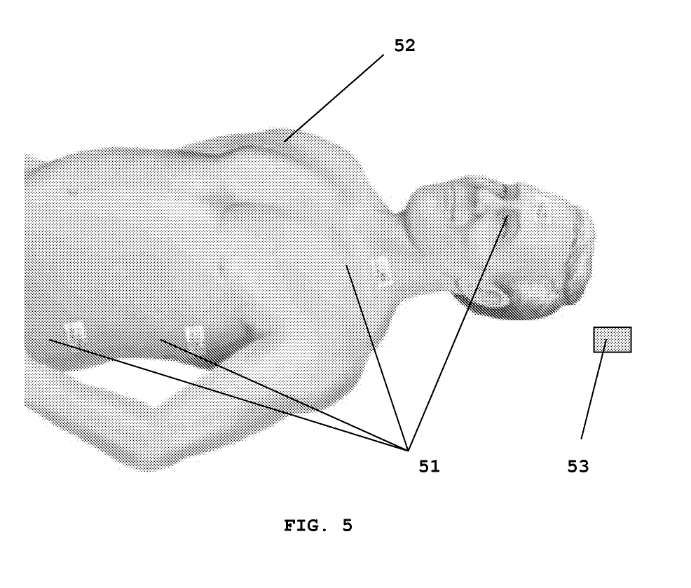

FIG. 5 shows a plurality of remote probes (51) positioned on a subject (52). It is particularly preferable that the programmable element is further programmed to analyze one or more remote probe data sets collected from a plurality of remote probes (51). The one or more remote probe data sets preferably comprise a measurement of differential displacements of the plurality of remote probes (51) in relation to one another. It is also preferable that the programmable element is further programmed to analyze an external probe data set collected from an external probe (53), and wherein a combination of one or more remote probe data sets and the external probe data set provides a measurement of differential displacements of the plurality of remote probes (51) in relation to the external probe (53).

Probes may be placed on thorax, abdomen, neck and extremities. Preferably in specified locations and more preferably in places specifically chosen by standardized measurements on thorax and abdomen. In another embodiment the probes have GPS like capabilities or functionality similar to an IR mouse where position is specifically reported to the device and incorporated in the data processing scheme. Preferably, there are a set of probes across the abdomen and thorax with additional probes on the neck and head. More preferably, there are four probes one on the forehead, one on the left neck in the supraclavicular region, one on the left lateral thorax and one on the left lateral lower abdomen. Preferably, the probes are tightly affixed to the body to prevent motion and to provide optimal contact for impedance measurements. In another embodiment, a net or array of sensors are placed around the body as part of a net or vest. FIG. 5 also shows external probe (53).

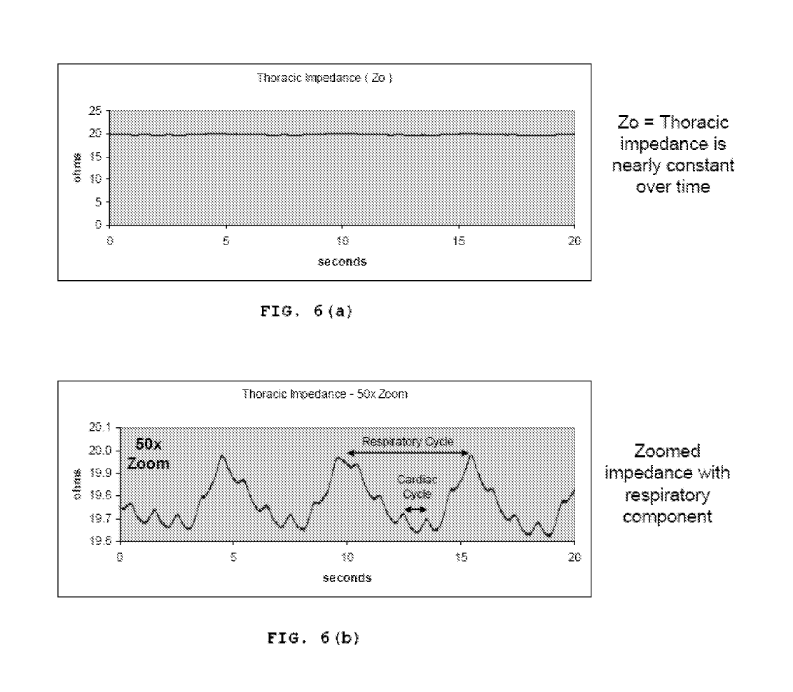

FIG. 6(a) shows a plot of Thoracic impedance (ohms) over time (seconds). As shown in FIG. 6(a) Thoracic impedance (Zo) appears to be nearly constant over time. However, FIG. 6(b) shows a plot of Thoracic impedance (ohms) over time (seconds) within a narrower range on the y-axis, i.e. the plot is “zoomed-in” with respect to Thoracic impedance. FIG. 6(b) demonstrates that upon closer inspection, a plot of Thoracic impedance versus time can provide information regarding respiratory cycle and cardiac cycle.

Other embodiments and uses of the invention will be apparent to those skilled in the art from consideration of the specification and practice of the invention disclosed herein. All references cited herein, including all patents and publications that are cited for any reason, including U.S. Provisional Application No. 60/973,292, on which priority is based, are specifically and entirely incorporated by reference. Furthermore, the term “comprising” includes the terms “consisting of” and “consisting essentially of.” The specification and examples should be considered exemplary only with the true scope and spirit of the invention embodied within the following claims.

1. A device for assessing a subject, the device comprising: at least one impedance measuring element functionally connected to a programmable element, programmed to analyze an impedance measurement, and to provide an assessment of at least one respiratory parameter of the subject.

2. A device according to claim 1, wherein the at least one impedance measuring element is one or more remote probes, wherein the one or more remote probes measure body wall movements, wherein the one or more remote probes are arranged as a net, vest, or array, and wherein one or more probes are placed on the thorax or abdomen of the subject.

3.-5. (canceled)

6. The device according to claim 1, wherein the at least one respiratory parameter is selected from the group consisting of the subject's respiratory rate, the subject's respiratory pressure, the subject's respiratory flow, the subject's end tidal CO₂, the subject's sublingual CO₂, intensity of respiration, variability of intensity of respiration, depth of respiration, variability of depth of respiration, localization of respiration, variation in localization of respiration, shape of a respiratory curve, change in shape of a respiratory curve, a respiratory curve based on inhaled volume, a respiratory curve based on exhaled volume, a respiratory curve based on inhaled pressure, a respiratory curve based on exhaled pressure, a respiratory curve based on inhaled flow, a respiratory curve based on exhaled flow, a respiratory curve based on motion of the subject's chest as measured by imaging, a respiratory curve based on motion of the subject's chest as measured by, contact sensors placed on the chest, and combinations thereof, and wherein the impedance measurement is based on a plurality of remote probe data sets, and wherein the programmable element is further programmed to enhance at least one of the plurality of remote probe data sets; or to stabilize at least one of the plurality of remote probe data sets; or to analyze each of the plurality of remote probe data sets for dynamic range and signal to noise ratio (SNR) values; or to evaluate and remove global motion; or to build a motion vector map to determine target displacements; or to calculate a differential motion map.

7.-8. (canceled)

9. The device according to claim 1, wherein the at least one respiratory parameter is selected from the group consisting of intensity of an acoustic signal, variability of an acoustic signal and is recorded for a duration of 30 seconds; continuously; intermittently; up to at least 10 of the subject's breaths; up to at least 100 of the subject'breaths; or up to at least 1000 of the subject's breaths.

10.-13. (canceled)

14. A device for assessing a subject, the device comprising:
at least one small-scale motion measuring element functionally connected to a programmable element, programmed to analyze a small-scale motion measurement and to provide an assessment of the subject based on the small-scale motion measurement, wherein the at least one small-scale motion measuring element comprises a high resolution lens functionally connected to a camera, and wherein the at least one small-scald motion measuring element comprises one or more remote probes.

15.-16. (canceled)

17. The device according to claim 14, wherein the one or more remote probes measure body wall movements, wherein the body wall movements are selected from the group consisting of movements of a thorax, a neck, an abdomen, an extremity, and combinations thereof.

18. (canceled)

19. The device according to claim 14, wherein the one or more remote probes measure impedance of the subject, and wherein the programmable element is further programmed to analyze an impedance measurement and to provide the assessment of the subject based on the small-scale motion measurement and the impedance measurement.

20. The device according to claim 14, wherein the at least one small-scale motion measuring element comprises one or more remote probes, and wherein the programmable element is further programmed to analyze one or more remote probe data sets collected from the one or more remote probes, wherein the one or more remote probe data sets comprise a measurement, selected from the group consisting of measurements of body wall movements of the subject, impedance of the subject, differential displacements of the one or more remote probes in relation to one another, and combinations thereof.

21. (canceled)

22. The device according to claim 20, wherein the programmable element is further programmed to analyze an external probe data set collected from an external probe, and wherein a combination of one or more remote probe data sets and the external probe data set provides a measurement of differential displacements of the one or more remote probes in relation to the external probe.

23. The device according to claim 22, wherein the measurement of differential displacements provides a respiratory rate of the subject.

24. The device according to claim 22, wherein the measurement of differential displacements is based on a plurality of remote probe data sets, and wherein the programmable element is further programmed to segment at least one of the plurality of remote probe data sets, or to enhance at least one of the plurality of remote probe data sets, or to stabilize at least one of the plurality of remote probe data sets, or to analyze each of the plurality of remote probe data sets for dynamic range and signal to noise ratio (SNR) values, or to evaluate and remove global motion or to calculate a differential motion map.

25. (canceled)

26. The device according to claim 22, wherein the measurement of differential displacements is based on a plurality of remote probe data sets, and wherein the programmable element is further programmed to build a motion vector map to determine target displacements.

27. (canceled)

28. A device for assessing a subject, the device comprising:
a high resolution lens functionally connected to
a camera functionally connected to
a programmable element, programmed to analyze at least one physiological parameter obtained by measuring differential displacements in a field of view of the camera.

29. The device according to claim 28, wherein the device is mounted on a stationary mount, or on a passively gyro-stabilized platform or is handheld or is stabilized by a gyrostabilizer.

30.-31. (canceled)

32. The device according to claim 28, wherein the device provides sensitivity to measure the differential displacements from a distance of up to 1 meter or up to 100 meters.

33. (canceled)

34. The device according to claim 28, wherein the at least one physiological parameter obtained by measuring differential displacements in a field of view of the camera is the heart rate of the subject, or the respiratory rate of the subject.

35. The device according to claim 28, wherein the measurement of differential displacements is based on a plurality of images, and wherein the programmable element is further programmed to segment at least one of the plurality of images; or to enhance at least one of the plurality of images; or to stabilize at least one of the plurality of images; or to analyze each of the plurality of images for dynamic range and signal to noise ratio (SNR) values, and to adjust camera gain and exposure based on the dynamic range and SNR values; or to evaluate and remove global image motion; or to build a motion vector map to determine target displacements; or to calculate a differential motion map.

36. The device according to claim 28, wherein the programmable element is further programmed to analyze one or more remote probe data sets collected from a one or more remote probes, wherein the one or more remote probe data sets comprise a measurement selected from the group consisting measurements of body wall movements of the subject; impedance of the subject differential displacements of the one or more remote probes in relation to one another, and combinations thereof.

37. (canceled)

38. The device according to claim 36, wherein the programmable element is further programmed to analyze an external probe data set collected from an external probe, and wherein a combination of one or more remote probe data sets and the external probe data set provides a measurement of differential displacements of the one or more remote probes in relation to the external probe and optionally provides a respiratory rate of the subject.

39.-40. (canceled)

41. The device according to claim 38, wherein the measurement of differential displacements is based on a plurality of remote probe data sets, and wherein the programmable element is further programmed to enhance at least one of the plurality of remote probe data sets; or to stabilize at least one of the plurality of remote probe data sets; or to analyze each of the plurality of remote probe data sets for dynamic range and signal to noise ratio (SNR) values; or to evaluate and remove global motion; or to build a motion vector map to determine target displacements; or to calculate a differential motion map or to segment at least one of the plurality of remote probe data sets.

42.-55. (canceled)
